(19) United States
(12) Patent Application Publication
(10) Pub. No.: US 2017/0149323 A1
(43) Pub. Date: May 25, 2017

(54) DRIVE UNIT WITH MAGNETIC INTERFACE

(71) Applicant: ABB GOMTEC GMBH, Seefeld (DE)

(72) Inventors: Bernd GOMBERT, Wörthsee (DE); Werner HOSL, Nabburg (DE); Patrick ROTHFUB, Hallbergmoos Erding (DE); Leopold KRAUSEN, München (DE)

(21) Appl. No.: 15/323,549

(22) PCT Filed: Jul. 2, 2015
(86) PCT No.: PCT/EP2015/001337

§ 371 Date: Jan. 3, 2017

(30) Foreign Application Priority Data
Jul. 4, 2014 (DE) 10 2014 009 892.3

(51) Int. Cl. H02K 49/10; A61B 34/30; H02K 7/00

(52) U.S. Cl. CPC H02K 49/104; H02K 7/003; A61B 34/30; A61B 2017/00477

(57) ABSTRACT

A drive unit (8) for driving a tool. The drive unit (8) has at least one first drive module (18) comprising a motor (12) and a wheel (32) driven in a rotary manner around an axis (16) by the drive module (18). The drive module (18) comprises a magnetic ring (22) surrounding the wheel (32), with which the wheel (32) is connected in a magnetically force-transmitting manner and with which the motor (12) is connected in a mechanically force-transmitting manner.

### TECHNICAL FIELD

The invention relates to a drive unit for a machine tool with a magnetic interface for driving and detachably coupling the machine tool to the drive unit.

### BACKGROUND INFORMATION

The patent application WO2007/075864 discloses a mechanical interface via which a surgical instrument can be operably coupled to a surgical robot. The interface has on the instrument side four rotatable bodies of revolution with which four rotatable bodies of revolution of complementary design on the robot side can be connected in a form-locking manner. The robot-side bodies of revolution can be driven by a drive unit integrated in the robot. Through the form-locking connection, a torque can be transmitted from each robot-side body of revolution to an instrument-side body of revolution.

However, when coupling the instrument to the robot it must be ensured that the corresponding bodies of revolution are aligned with one another and are not twisted. Otherwise the coupling of the instrument is obstructed. The bodies of revolution must therefore either be oriented with respect to each other manually by the user, or an additional mechanism must be provided allowing an automatic orientation of the bodies of revolution.

The present invention is thus based on solving the problem of creating a drive unit with a simplified interface for coupling a tool in which bodies do not have to be oriented with respect to each other for form-locking connection.

This problem is solved through a drive unit with at least one first drive module comprising a motor, and a first wheel driven in a rotary manner around an axis by the drive module, wherein the drive module comprises a magnetic ring surrounding the first wheel, with which the first wheel is connected in a magnetically force-transmitting manner and with which the motor is connected in a mechanically force-transmitting manner.

### SUMMARY

An electric motor is preferably chosen as the motor. The motor drives the magnetic ring via the mechanically force-transmitting connection, in which the driving force or the torque of the motor is transmitted through a mechanical contact between two components of the connection. Form-locking connections, for example a gear drive, and frictional connections, for example a belt drive, can be used as a mechanically force-transmitting connection which transmits a driving force or a torque of the motor to the magnetic ring and causes this to rotate around an axis.

In contrast, the magnetically force-transmitting connection transmits a torque from the magnetic ring to the first wheel surrounded by the magnetic ring in a contact-free manner. Since the first wheel is also mounted rotatably around the axis, due to the magnetic interaction it is carried along by the rotating magnetic ring and is consequently driven in a rotary manner. Consequently the transmission of a driving force to the first wheel is possible without a form-locking connection.

In order that the magnetic ring can engage in a magnetic interaction with the wheel, the magnetic ring is equipped on its inner circumference with a plurality of permanent magnets which engage in a magnetic traction with the wheel. Conversely, a plurality of permanent magnets can be distributed on the outer circumference of the wheel which create a traction with the magnetic ring. The permanent magnets advantageously magnetize ferromagnetic bodies which are distributed around the circumference of the component corresponding with the wheel or magnetic ring equipped with permanent magnets.

The magnets adjacent to a central magnet advantageously have an opposite polarity to the central magnet in order to obtain a plurality of magnetic fields encircling the circumference of the wheel.

The mechanically force-transmitting connection between motor and magnetic ring advantageously comprises a gear. The gear is in particular designed as a worm gear, which allows a high gear ratio. Herein, the motor can drive a worm which meshes with a gearing arranged on the outer circumference of the magnetic ring.

The drive unit can comprise at least two drive modules which each have a magnetic ring driven in a rotary manner around an axis by a motor. The drive modules can be arranged with their axes coaxial to one another. The magnetic ring of the at least second drive module can surround a second wheel, and in order to drive this wheel the magnetic ring can be connected with the wheel in a magnetically force-transmitting manner.

Preferably, the drive modules are of identical design, allowing the number of identical parts to be increased so as to make manufacture economical.

A wheel driven by a drive module can be used to drive further components. For example, the first wheel can be connected with a shaft. If the drive unit includes at least a second wheel, which can be driven by a second drive module, the shaft can extend through the second wheel.

The second wheel can be connected with a shaft sleeve which surrounds the shaft and is mounted so as to be moveable around it. In this way, the drive forces of the first and of the second wheel are accessible and can be picked up from a single drive side of the drive unit, i.e. the drive unit only requires one output via which the drive forces of the two wheels can be passed out of the drive unit.

In principle, different connections can be chosen in order to connect the shaft with the first wheel. According to a first variant, the shaft is connected with the wheel, rotationally fixed in a form-locking manner and yet axially moveable, for example by means of a tongue-and-groove connection. This allows a torque to be transmitted from the wheel to the shaft, whereas the shaft can be moved freely in an axial direction relative to the wheel.

According to a second variant, the connection of the shaft with the wheel is designed in the form of a screw thread. This allows a rotary movement of the wheel to be translated into an axial travel movement of the shaft.

According to a third variant, the first and the second variant are combined with one another such that the drive unit comprises two drive modules which each drive a wheel, wherein the shaft is connected with one of the two wheels in a rotationally fixed and axially moveable manner and is connected with the other wheel by means of a screw thread. This means that the shaft can be adjusted rotationally through one wheel and in an axial direction through the other wheel.

In order to achieve a compact arrangement in cases where a plurality of drive modules are built into a drive unit, the drive modules can be arranged at intervals along a common axis.

A magnetic ring and a wheel surrounded by it are preferably separated by an air gap or intermediate space via which the magnetic forces can be transmitted. If a plurality of drive modules are arranged at intervals, then the intermediate spaces of the individual drive modules are preferably designed to align axially with one another. For this purpose, the outer diameters of the respective wheels and the inner diameters of the respective magnetic rings can for example in each case be identical in size.

A barrier impermeable to germs, for example in the form of a sleeve, can extend through the intermediate spaces. The sleeve is preferably made of a non-magnetisable material to prevent a magnetic connection with the magnetic ring or the wheel. However, the sleeve permits the magnetically force-transmitting connection between magnetic ring and wheel.

The property of impermeability to germs fulfils a protective function against a contamination of a working space which needs to be kept sterile. This means that the drive unit can for example be used in an operating theatre in order to drive a surgical tool.

Each drive module preferably includes a mounting segment, in which the magnetic ring of the drive module is held by at least one roller bearing. The mounting segment can be firmly connected with a housing of the drive unit. This allows the magnetic rings to be mounted so as to be rotatable around the axis relative to the housing of the drive unit.

The magnetic ring or rings preferably carry a gear ring, the outer diameter of which is larger than that of the roller bearing. This means that, for example, a worm of a mechanically force-transmitting connection designed in the form of a worm gear can be arranged on the outer circumference of the magnetic ring and can mesh with the gear ring of the magnetic ring. In addition, a large gear ring makes possible a high gear ratio of the gear.

The mounting segments of a plurality of drive modules can be plugged together with one another. Such a plugged connection simplifies assembly and makes possible a fixed connection of all drive modules relative to the housing of the drive unit.

The wheels which are surrounded by the magnetic rings can be connected to form an assembly which is accommodated removably in the magnetic rings. The assembly can for example represent an operating unit of a tool in which the rotatable wheels are used as a control drive for the control of certain tool functions, for example the actuation of an end effector located on the tool.

The magnetically force-transmitting connection between the wheels and the corresponding magnetic rings makes possible a simple change of the assembly or of the tool, since the assembly can be inserted into or removed from the magnetic rings without needing to pay attention to the orientation of the wheel in relation to the magnetic rings because, in contrast to a form-locking connection, no mutual orientation of the force-transmitting elements, i.e. in this case wheel and magnetic ring, is necessary.

The wheels are preferably connected with one another in a rotatable and axially immovable manner through roller bearings. This makes possible a simple structure in which the wheels, analogously to the magnetic rings, are arranged at staggered intervals and are rotatable relative to one another around a common longitudinal axis, in particular to form an assembly.

The assembly can include two contact elements between which the wheels are arranged, and which are fixed radially to a housing accommodating the magnetic rings. These contact elements can be conical in form and can be supported on correspondingly formed contact surfaces in the housing. This means that the wheels of the assembly are mounted in the housing coaxially with the common axis of the magnetic rings and maintain around their circumference a constant air gap with respect to their corresponding magnetic ring.

### BRIEF DESCRIPTION OF THE DRAWINGS

These and other features and advantages of the present invention will be understood by reading the following detailed description, taken together with the drawings wherein:

FIG. 1 shows a robot fitted with an instrument;

FIG. 2 shows a cross section through a drive unit with an inserted instrument;

FIG. 3 shows a cross section through the drive unit without the instrument;

FIG. 4 shows the instrument;

FIG. 5 shows a cross section through a drive module of the drive unit;

FIG. 6 shows an operating unit at the proximal end of the instrument in cross section;

FIG. 7 shows a distal end of the instrument with a swivel mechanism and an end effector in extended position;

FIG. 8 shows the distal end of the instrument from FIG. 7 in angled position;

FIG. 9 shows the distal end of the instrument in cross section;

FIG. 10 shows an overview in table form of the actuation possibilities of the instrument;

FIG. 11 shows the distal end of the instrument with grippers of the end effector in opened position;

FIG. 12 shows the distal end of the instrument with the end effector rotated relative to the swivel mechanism;

FIG. 13 shows the distal end rotated around the longitudinal axis of the instrument;

FIG. 14 shows a distal end with a second embodiment of the swivel mechanism;

FIG. 15 shows a distal end with a third embodiment of the swivel mechanism; and

FIG. 16 shows a distal end with a fourth embodiment of the swivel mechanism.

### DETAILED DESCRIPTION OF THE PREFERRED EMBODIMENTS

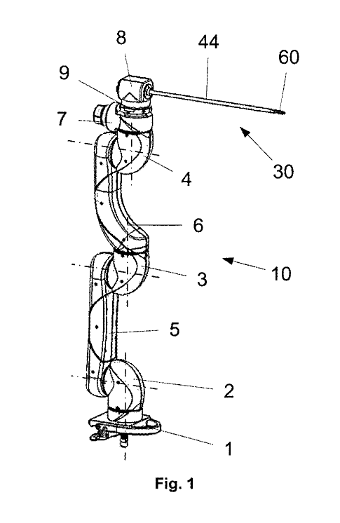

FIG. 1 shows a robot 10 and an instrument 30 coupled to the robot 10. The robot 10 comprises a fixing element 1 which serves to fix the robot 10 to any suitable object. Connected to the fixing element 1 is an articulated joint 2 which rotatably connects an arm element 5 with the fixing element 1. A second arm element 6 is rotatably connected with the arm element 5 via an articulated joint 3. Connected to the arm element 6 via a further articulated joint 4 is an input device 7 which makes it possible for the user to control the robot 10 and/or the instrument 30.

Each of the three articulated joints 2, 3 and 4 has two axes of rotation oriented perpendicular to one another, so that a rotational movement is possible on two connection sides of an articulated joint. This means that the robot 10 can be moved in six degrees of freedom. In order to allow corresponding control of the robot 10, the input device 7 preferably has a cap which can also be moved manually in six degrees of freedom. A more detailed explanation of such a robot control device can be found in the applicant's as yet unpublished patent application DE 102013019869.

A distal end of the robot 10 is formed by a drive unit 8 which is connected firmly with the input device 7 via a flange 9. The instrument 30 can be coupled exchangeably with the drive unit 8 and can be driven or actuated by the drive unit 8.

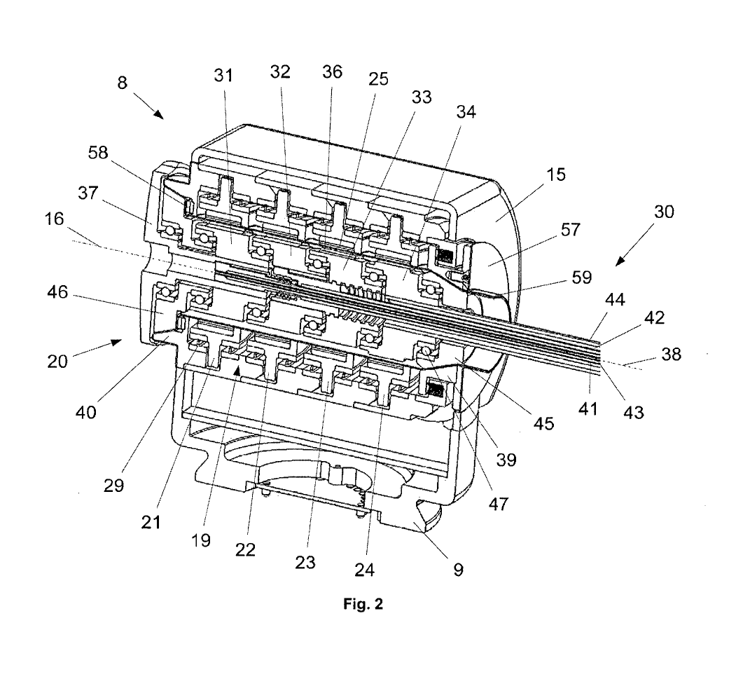

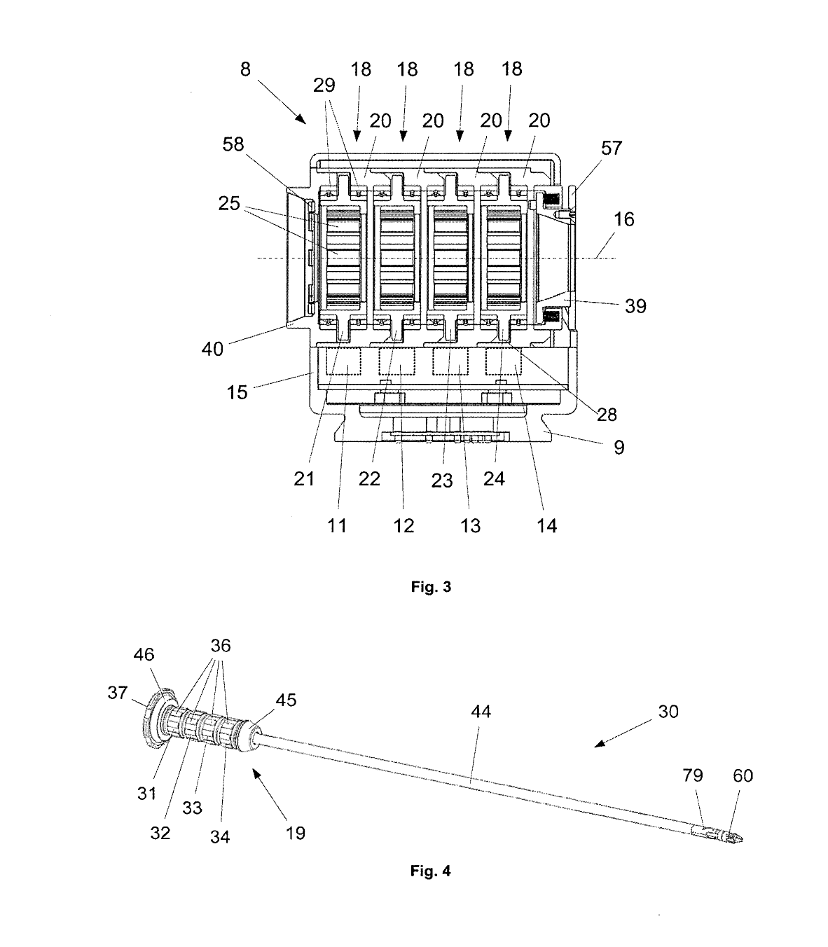

FIG. 2 shows a cross-sectional view of the drive unit 8 with the inserted instrument 30, FIG. 3 shows a cross-sectional view of the drive unit 8 without the instrument, and FIG. 4 shows the instrument 30 detached from the drive unit 8.

The instrument 30 has an operating unit 19 with four wheels 31, 32, 33 and 34, a base element 46 adjacent to the left next to the left-hand outer wheel 31 and a contact element 45 adjacent to the right next to the right-hand outer wheel 34. The wheels 31, 32, 33 and 34 can rotate relative to one another and relative to the base and the contact elements 45, 46 in order to drive movements of an end effector 60 connected by means of a swivel mechanism 79 with a shaft sleeve 44. The base element 46 and the contact element 45 are shaped so as to taper conically in the direction of the end effector 60.

The drive unit 8 has a housing 15 which is firmly connected with the flange 9. The drive unit 8 is hollow throughout along an axis 16, so that in order to couple the instrument 30 with the drive unit 8 the instrument 30 can be introduced from one side into the drive unit 8 along the axis 16.

In the coupled state of the instrument 30, the contact element 45 comes to rest against a correspondingly formed limit stop 39 in the housing 15 of the drive unit 8. The limit stop 39 is spring-mounted in the housing 15 and exerts a preload force on the instrument 30.

The side of the housing 15 opposite the limit stop 39 has a further limit stop 40 against which the base element 46 of the instrument 30 comes to rest in the coupled state. The limit stop 40 is preferably also conically formed, correspondingly to base element 46.

The limit stops 39 and 40 prevent the instrument 30 from slipping through in an axial direction. A defined plug-in position of the instrument 30 in an axial direction and in a radial direction starting out from the axis 16 is determined through the conical design of the two limit stops 39 and 40 as well as of the contact and base elements 45 and 46 of the instrument 30. As FIG. 2 shows, this allows a coaxial alignment of a longitudinal axis 38 running through the instrument 30 with the axis 16 running through the drive unit 8.

A holding element 58 is preferably provided on the housing 15 which detachably fixes the instrument 30 with the housing 15 in order, in the coupled state, to prevent the base element 46 from rotating relative to the housing 15 or from axially slipping within the drive unit 8 along the axis 16. The holding element 58 can comprise a magnet which exerts a holding force on the base element 46, which consists of a ferromagnetic material.

Four identical drive modules 18 are built into the drive unit 8. The first drive module comprises a magnetic ring 21 driven by a motor 11, the second drive module comprises a magnetic ring 22 driven by a motor 12, the third drive module comprises a magnetic ring 23 driven by a motor 13 and the fourth drive module comprises a magnetic ring 24 driven by a motor 14. The magnetic rings each comprise a hollow-cylindrical inner section equipped with magnets 25 and an outer section in the form of a gear ring 28 projecting radially from the inner section. All four magnetic rings 21, 22, 23 and 24 are mounted in the housing 15 within each case at least one roller bearing 29, in this case with two roller bearings 29 on each side of the outer section.

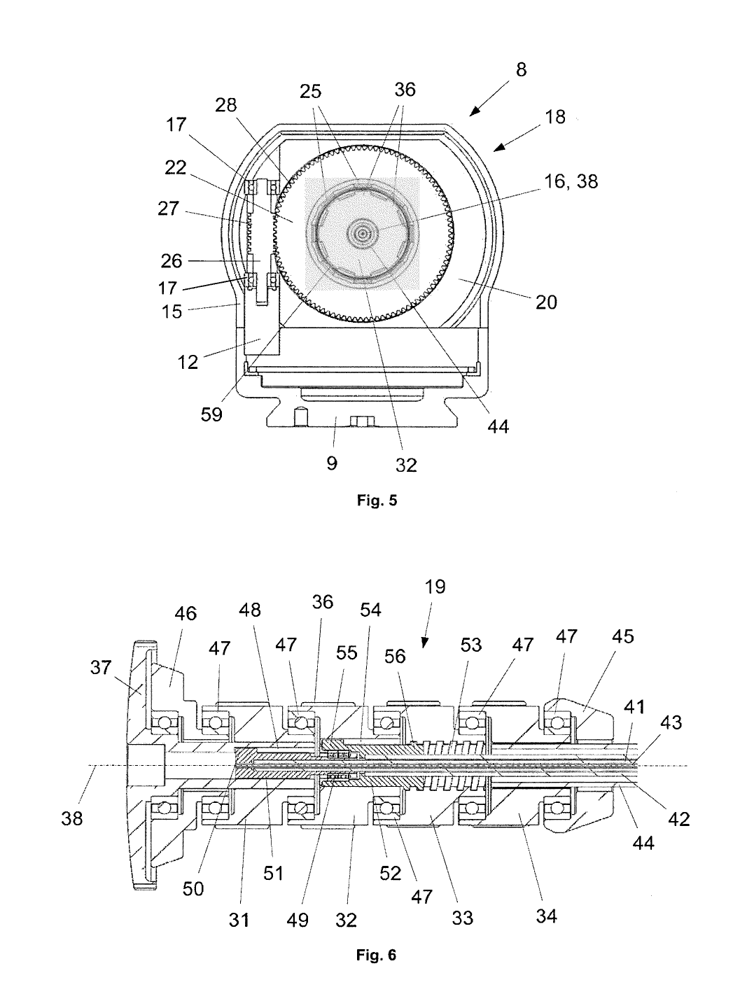

To represent all four drive modules 18, FIG. 5 shows their structure and functional principle with reference to the example of the second drive module 18. The drive module 18 has a stable mounting segment 20. The motor 12 is firmly connected with the mounting segment 20 and drives a gear 26.

The gear 26 is in this case designed as a worm gear and has a worm 27 which meshes with the gear ring 28. The worm 27 is mounted rotatably relative to the mounting segment 20 by means of bearings 17 and transmits the torque generated by the motor 12 to the magnetic ring 22 in order to drive this around the axis 16 in a rotational manner. The magnetic ring 22 thus functions as a worm wheel and is connected in a mechanically force-transmitting manner with the motor 12.

As can be seen in FIG. 3, the individual drive modules 18 are plugged together with one another via their mounting segments 20 in that each mounting segment 20 has, on its right side in FIG. 3, a projection which engages in a complementary recess in the mounting segment 20 adjacent to the right, so that the gear rings 28 are sandwiched to the right and left by different mounting segments 20. On the one hand, the plugged connection allows a modular-type structure and a fixed alignment between the mounting segments 20. On the other hand, the mounting segments 20 serve the purpose of attachment to the housing 15 of the drive unit 8, with which they are for example screwed together, or with which they can also be plug-connected.

The four drive modules 18 are arranged next to one another and aligned coaxially with one another, so that each magnetic ring 21, 22, 23 and 24 can rotate around the common axis 16. Motors of the four drive modules 18 can be controlled individually, so that the magnetic rings 21, 22, 23 and 24 can be set in rotation independently of one another.

If a magnetic ring 21, 22, 23, 24 rotates, the magnets 25 fixed to the relevant magnetic ring rotate with it. Permanent magnets are preferably used as magnets 25. Alternatively, electromagnets can also be used.

The four wheels 31, 32, 33, 34 of the operating unit 19 of the instrument 30 are arranged concentrically around the longitudinal axis 38 of the instrument 30 and, when an instrument 30 is coupled with the drive unit 8, each of them is surrounded by a magnetic ring 21, 22, 23, 24, that is to say the magnetic ring 21 is arranged concentrically around the wheel 31, the magnetic ring 22 is arranged concentrically around the wheel 32 and so forth (see FIGS. 2 and 4).

Each wheel 31, 32, 33, 34 has on its circumference a driving-force-transmitting structure in the form of a plurality of ferromagnetic bodies 36 which engage in a magnetic traction with the magnets 25. The motor-driven magnetic rings 21, 22, 23 and 24 therefore serve, on the one hand, to allow detachable coupling of the instrument 30 with the drive unit 8 and on the other hand for the transmission of torques to a wheel 31, 32, 33 and 34 of the operating unit 19 of the instrument 30 corresponding to the respective magnetic ring 21, 22, 23 and 24. In other words, each magnetic ring 21, 22, 23, 24 is connected in a magnetically force-transmitting manner with a corresponding wheel 31, 32, 33, 34.

FIG. 6 shows the operating unit 19 of the instrument 30 in cross section. Pairs of the four wheels 31, 32, 33, 34 are in each case connected with one another via a roller bearing 47 so as to be rotatable around the longitudinal axis 38 and are arranged next to one another at fixed intervals. The left-hand outer wheel 31 is supported rotatably on the base element 46 by a bearing 47 pressed onto the base element 46. The right-hand outer wheel 34 is supported on the contact element 45 by a bearing 47 pressed into the contact element 45.

In the case of the bearings 47 arranged between two wheels 31, 32, 33, 34, an outer ring of the bearing 47 is pressed into one of the wheels 31, 32, 33, 34 and an inner ring of the bearing 47 is pressed onto the other wheel 31, 32, 33, 34.

The bearings 47 arranged one each side of the wheels 31, 32, 33, 34 ensure an axial cohesion of the construction elements connected by the bearings 47.

As shown in FIG. 6, the ferromagnetic bodies 36 can overlap in the axial direction of the bearing 47 in order to make optimal use of the surface area available on the circumference of a wheel.

The wheel 32 adjacent to the left-hand wheel 31 is connected non-rotatably with a first shaft 42. The non-rotatable connection is in the form of a tongue-and-groove connection with a tongue 55 connected with the first shaft 42 and a groove 54 recessed in the wheel 32 and makes possible an axial relative movement as well as a transmission of a torque between the first shaft 42 and the wheel 32. The tongue 55 can as in this case form part of a right-hand sleeve 52 with which the first shaft 42 is firmly connected. Instead of the tongue-and-groove connection, a splined shaft connection for example could also be chosen.

The first shaft 42 engages by means of an outer thread 56 in an inner thread 53 of the wheel 33 adjacent to the right-hand wheel 34. The outer thread 56 is located on the sleeve 52 connected firmly with the first shaft 42.

The outer thread 56 and the inner thread 53 form a screw thread which transforms a rotary movement of the second wheel 33 into a translation movement of the first shaft 42 along the longitudinal axis 38. The pitch of the thread determines the core/thread ratio and thus the advance per rotation.

The difference in the lengths of groove 54 and tongue 55 determines the axial freedom of movement of the first shaft 42. Alternatively, other rotation-translation conversion gear mechanisms can be chosen, for example a ball screw drive.

The two wheels 32, 33 can co-operate so that on rotation of one of the two wheels 32, 33 the first shaft 42 performs a translatory or axial movement along the longitudinal axis 38 and on simultaneous rotation of both wheels 32, 33 it performs a rotary movement around the longitudinal axis 38.

The wheel 34 is firmly connected with the shaft sleeve 44 which is arranged coaxially with the first shaft 42 and surrounds this. Through rotation of the third wheel 34 the shaft sleeve 44 is driven and rotates relative to the first shaft 42 around the longitudinal axis 38. The end effector 60 which is connected with the shaft sleeve 44 by means of the swivel mechanism 79 is also thereby rotated around the longitudinal axis 38.

Within the first shaft 42, which in this case is hollow throughout, a second shaft 41 is arranged coaxially with the longitudinal axis 38. The second shaft 41 is connected with the first shaft 42 in a rotatable and axially fixed manner by means of a (roller) bearing 49, i.e. a relative movement between the first and second shaft 41, 42 is only possible through a rotary movement, but not through an axial movement. The second shaft 41 can thus rotate relative to the first shaft 42 around the common longitudinal axis 38 and in the event of an axial movement of the first shaft 42 it is carried along by the latter, so that the second shaft 41 always moves together with the first shaft 42 in an axial direction, but can rotate independently of it.

The second shaft 41 is connected non-rotatably with the wheel 31. The non-rotatable connection is in the form of a tongue-and-groove connection with a tongue 50 connected with the second shaft 41 and a groove 48 recessed in the wheel 31, and makes possible an axial relative movement as well as a transmission of a torque between the second shaft 41 and the wheel 31. Insofar as, during an axial movement of the first shaft 42, the second shaft 41 is carried along with this, the second shaft 41 can move freely in the wheel 31 in an axial direction.

The tongue 50 can as in this case form part of a left-hand sleeve 51 with which the second shaft 41 is firmly connected. Instead of the tongue-and-groove connection, a splined shaft connection for example could also be chosen. The difference in the lengths of groove 48 and tongue 50 determines the axial freedom of movement of the second shaft 41. Since the first and second shaft move together in the axial direction, the difference in length of groove 48 and tongue 50 is equal to the difference in length of groove 54 and tongue 55.

The end effector 60 located on the distal end of the instrument 30 is swivelably connected with the shaft sleeve 44 via a swivel mechanism 79. The swivel mechanism 79 comprises a proximal member 61 which is firmly connected with the shaft sleeve 44. In a further development of the invention, the proximal member 61 and the shaft sleeve 44 can be formed as a single part.

A distal member 62 of the swivelling mechanism 79, which is coupled to a base 63 of the end effector 60, is swivelably connected to the proximal member 61.

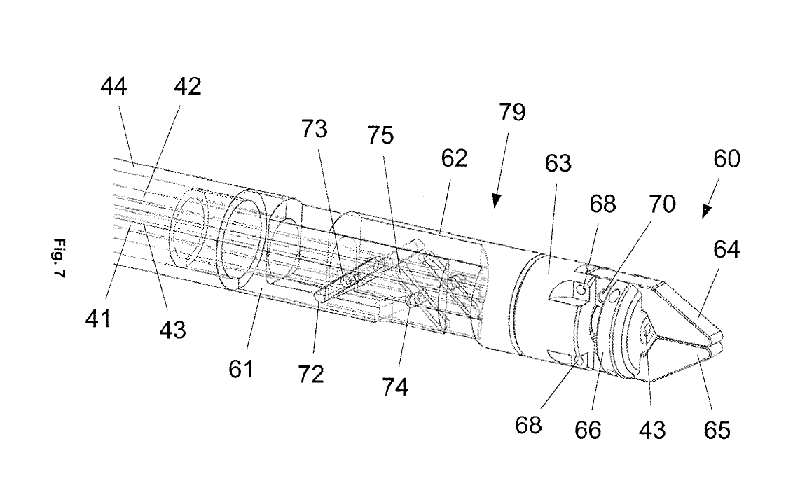

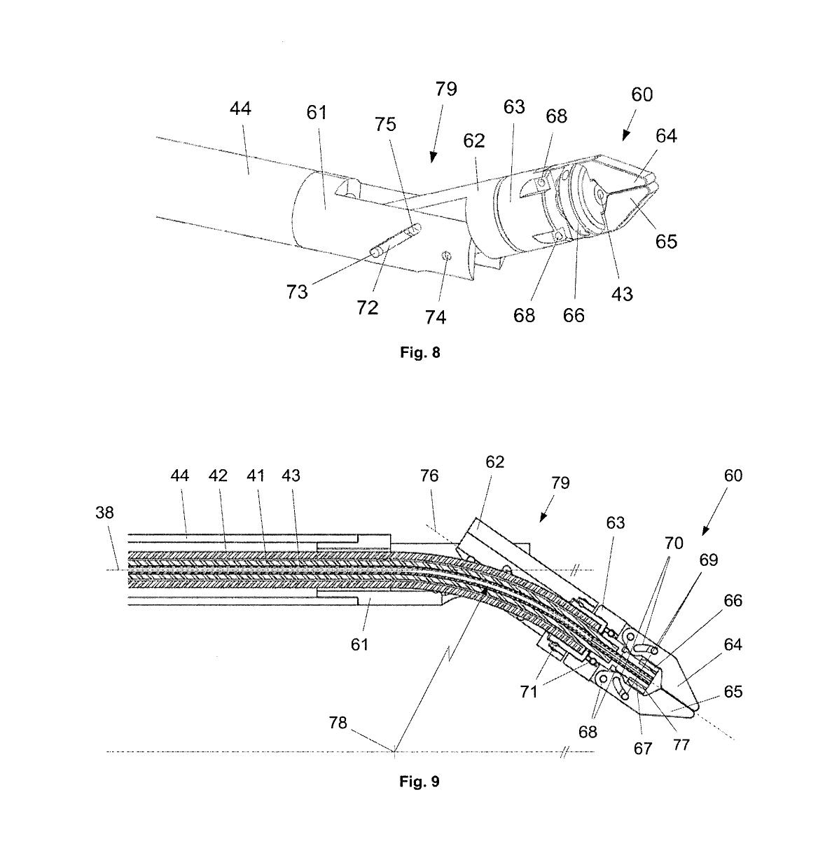

The swivelable connection of the proximal and distal members 61 and 62 can be formed by any design of swivel bearing in which the proximal member 61 serves as thrust bearing for the distal member 62. As FIG. 7 (with concealed edges) and FIG. 8 (without concealed edges) show, in the present exemplary embodiment a slotted guide system was chosen as swivel bearing in which a guide slot 72 is formed in the proximal member 61 and a guide slot 75 is formed in the distal member 62.

A guide slot 72, 75 of one member 61, 62 interacts with a bolt 73, 74 fixed to the other member 62, 61 in that the course of the guide slot 72, 75 serves as a guide for the bolt 73, 74. At least one of the guide slots 72, 75 has a course running non-parallel to the longitudinal axis 38 of the instrument 30. The course is preferably linear, but can alternatively also be curved.

In the event of a relative movement of the distal member 62 the bolts 73, 74 guided in the guide slots 72, 75 follow the course of the guide slots and cause the distal member 62 to swivel accordingly, whereby an end effector axis 76 extending lengthways through the end effector 60 is angled relative to the longitudinal axis 38 of the instrument 30. As shown in FIG. 9, the swivelling movement takes place around a swivel axis 78 oriented normally to the longitudinal axis 38. The end effector 60 coupled to the distal member 62 swivels along with this accordingly.

The end effector 60 can swivel in the direction shown in FIG. 9 or in a direction opposite to this (as shown in FIG. 8). The swivelling movement in one direction or in the opposite direction both take place around a swivel axis oriented normally to a parallel of the longitudinal axis 38. In FIG. 9 the end effector 60 swivels around the swivel axis 78, in FIG. 8 around a swivel axis (not shown) running at a distance from the swivel axis 78 and parallel thereto.

In an alternative embodiment of the invention, the swivel mechanism can be realised with only a single slotted guide system in which a guide slot is recessed either in the proximal member or in the distal member and interacts with a bolt on the other member and the bolt has a cross section which is elongated in the direction of the guide slot which engages non-rotatably in the guide slot.

The first shaft 42 and the second shaft 41 have at least one flexible partial region. This partial region extends through the swivel mechanism 79 and makes it possible, in the event of a swivelling movement of the distal member 62, for the first shaft 42 and the second shaft 41 to swivel along with it accordingly. The flexible partial region is preferably elastically deformable in both shafts 41, 42.

As FIG. 9 shows, the distal end of the first shaft 42 is firmly connected with the base 63 of the end effector 60. This means that the base 63 of the end effector 60 can be moved by means of the first shaft 42. If the first shaft 42 is driven in a rotary manner, then the base 63 is rotated around the end effector axis 76 relative to the swivel mechanism 79.

If the first shaft 42 is driven in an axial direction, then the base 63 of the end effector 60 is moved in an axial direction, whereby the distal member 62 of the swivel mechanism 79 connected with the base 63 is at the same time slid along the guide slot 72 or 75 and performs a swivelling movement around the swivel axis 78, i.e. the end effector 60 can be swivelled through an axial movement of the first shaft 42.

If the shaft sleeve 44 is driven in a rotary manner, the swivel mechanism 79 rotates together with the end effector 60 around the longitudinal axis 38.

The end effector is designed according to the intended purpose of the instrument 30 (for example an industrial or surgical application) and comprises, for example, a camera, a light source, a blade, a welding electrode or any other type of tool. In the present exemplary embodiment the end effector 60 is designed as a gripper tool and has two grippers 64 and 65 which are both connected with the base 63 so as to rotate around a gripper axis 68.

The base 63 is connected with the distal member 62 of the swivel mechanism 79 by means of a bearing 71 so as to rotate around an end effector axis 76 running through the distal member 62 and the base 63.

Both grippers 64 and 65 are connected with an actuating member 66. The connection is designed as a slotted guide system in which preferably each gripper 64 and 65 has a guide slot 70 and the actuating member 66 carries the corresponding bolt 69. Alternatively, the reverse arrangement could be chosen.

The actuating member 66 is mounted so as to be axially displaceable along the end effector axis 76. The movement of the actuating member 66 is driven by the second shaft 41. For this purpose, a drive element 77 is attached on the distal end of the shaft 41 which engages with the actuating member 66 by means of a screw thread 67. The screw thread 67 translates a rotary movement of the second shaft 41 into an axial movement of the actuating member 66 along the end effector axis 76.

As a result of a displacement of the actuating member 66, the bolts 69 are moved along the end effector axis 76 and slide along the path defined by the guide slots 70. The bolts 69 thereby press laterally against the guide slots 70, so that depending on the direction of movement of the actuating member 66 the grippers 64 and 65 are spread or are pinched together. Advantageously, the guide slots 70 are formed such that the grippers 64 and 65 are pressed together when the actuating member 66 is moved away from the base 63 and that the grippers 64 and 65 are spread when the actuating member 66 is moved towards the base 63 in order that the forces acting from the bolt 69 on the grippers 64, 65 when closing the grippers 64, 65 are translated into the greatest possible clamping forces.

The guide slot 70 associated with a gripper 64, 65 and its gripper axis 68 are arranged such that the gripper axis 68 runs outside of the guide slot 70 of the slotted guide system. This prevents the bolt 69 guided in the respective guide slot 70 of the gripper 64, 65 from being able to assume a position which coincides with the gripper axis 68 of the gripper 64, 65, i.e. the gripper axis 68 and bolt 69 are always spaced apart from one another, so that the force acting on the bolt always generates a torque around the gripper axis 68.

As shown in FIG. 9, the guide slot 70 can be located next to a plane which is oriented perpendicular to the end effector axis 76 in which the gripper axes 68 of the grippers 64, 65 run without intersecting this plane. In the present exemplary embodiment the guide slot 70 runs between this plane and a clamping zone or the tip of the respective gripper 64, 65, in order to the make the best possible use of the available construction space for the grippers 64, 65.

In order for a greatest possible torque to be applied to the grippers 64, 65 when clamping them, in the closed state of the grippers 64, 65 the bolts 69 must assume a position in the guide slots 70 in which there is a maximum distance between the bolt 69 and the gripper axis 68 of a gripper 64, 65. For this purpose, the guide slots 70 of each gripper 64, 65 are designed such that the distance between an end of the guide slot 70 facing the gripper axis 68 and the end effector axis 76 is less than the distance between an end of the guide slot 70 facing away from the gripper axis 68 and the end effector axis 76. In this case the grippers 64, 65 are closed when the bolts 69 are moved away from the gripper axes 68 and towards the clamping zone of the grippers 64, 65.

In order to provide the end effector 60 with good stability as well as making it compact, a cut-out 80 is provided in the actuating member 66 for each gripper 64, 65, as shown in FIG. 11. On the one hand, the bolts 69 are held on both sides of the respective cut-out 80 in the actuating member 66, so that the cut-outs 80 form an accommodation for the bolts 69. On the other hand, in the closed state the grippers 64, 65 can be supported against a lateral contact surface of the cut-out 80. This prevents the grippers 64, 65 from bending away to the side when holding a heavy load. In addition, this accommodation of the grippers 64, 65 prevents the bolts 68 from slipping out of their guide slots 70.

A continuous channel 43 can be integrated within the instrument 30 which can be used to conduct media, for example in order to rinse the end effector 60 or the object to be gripped by the end effector 60 or in order to conduct gas. The channel 43 is preferably formed through a cavity in the second shaft 41, as shown in FIGS. 6 and 9.

The instrument 30 can also have a handle 37 at the proximal end which is connected non-rotatably with the second shaft 41 (see FIGS. 4 and 6). This handle 37 can be used in order to insert the instrument 30 in the drive unit 8 or remove it. The second shaft 41 can be actuated through manual rotation of the handle 37, thereby controlling the grippers as explained above. This makes it possible for the user to open the grippers 64, 65 manually via the drive unit 8 in the event of a failure of the motorized drive function.

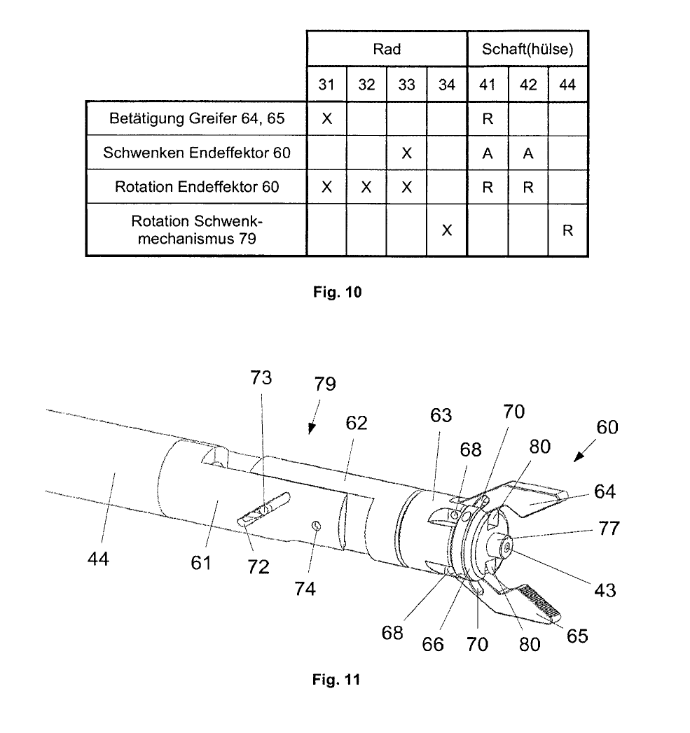

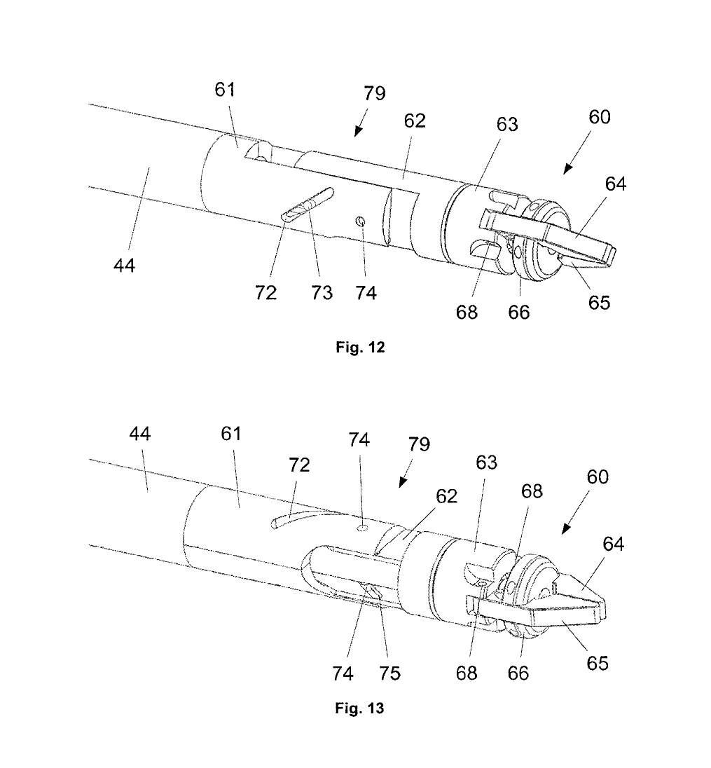

FIG. 10 lists in table form the individual actuation possibilities and illustrates once again the functional principle of the wheels 31, 32, 33 and 34, the shaft sleeve 44 and the shafts 41 and 42 as well as their effects on the actuation of the end effector 60. A distinction is made between the following actuations: actuation of the grippers 64, 65 (see FIG. 11); swivelling of the end effector 60 around the swivel axis 78 (see FIGS. 8 and 9); rotation of the end effector 60 around the end effector axis 76 (see FIG. 12) and rotation of the swivel mechanism 79 together with the end effector 60 around the longitudinal axis 38 (see FIG. 13). The wheels which necessarily need to be driven in order to perform the relevant actuation are marked with an “X”. The movements of the shaft sleeve and the shafts caused through the driven wheels are marked with an “R” or with an “A”, wherein “R” defines a rotational movement and “A” defines an axial movement.

Accordingly, the second shaft 41 is rotated through rotation of the fourth wheel 31 on its own. The direction of rotation of the second shaft 41 determines whether the actuating member 66 is moved towards the base 63 or away from it and whether, accordingly, the grippers 64 and 65 are forced to spread or close together.

The first shaft 42 is moved in an axial direction through rotation of the second wheel 33. The second shaft 41 is carried along by the first shaft 42 and is thus also moved in an axial direction. The axial movement of the first shaft 42 causes a displacement of the base of the end effector 60, which is superimposed on a swivelling movement around the swivel axis 78 of the distal member 62 of the swivel mechanism 79 which is connected with the base 63.

In order to rotate the end effector 60 relative to the swivel mechanism 79 around the end effector axis 76, the first shaft 42 is set into rotation through synchronous rotation of the first and second wheels 32 and 33. In order to avoid a travel movement of the actuating member 66 caused by the difference in the speed of rotation between the first and second shafts 41, 42 which would trigger an actuation of the grippers 64 and 65, the second shaft 41 is also rotated synchronously with the first shaft 42 by driving the fourth wheel 31.

The shaft sleeve 44 and thus the swivel mechanism 79 connected with it are rotated around the longitudinal axis 38 by driving the third wheel 34. In order also to rotate the end effector 60 together with the swivel mechanism 79, all the wheels 31 to 34 can be driven simultaneously, so that the two shafts 41 and 42 rotate together with the shaft sleeve 44.

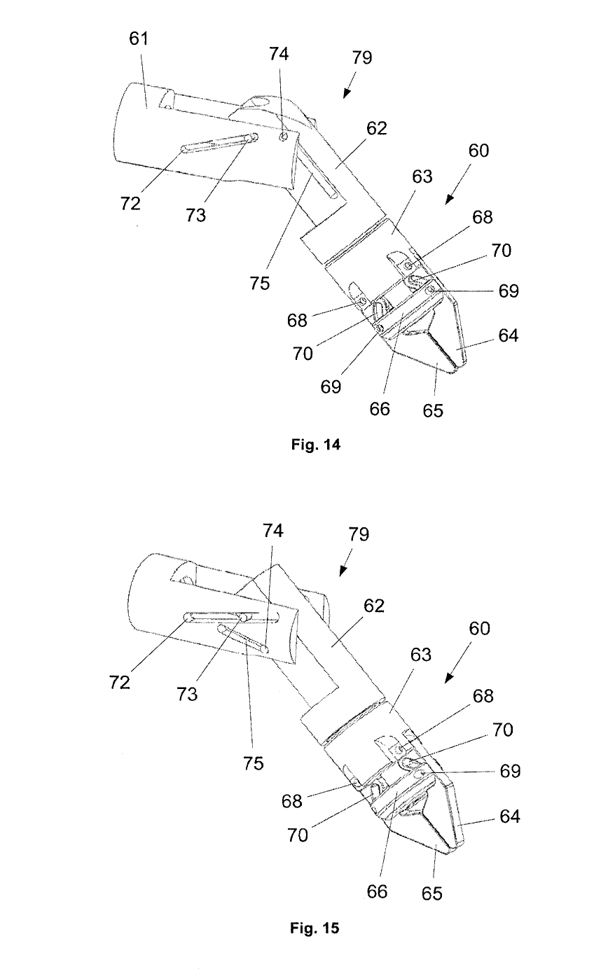

FIGS. 14 to 16 show alternative embodiments of the swivel mechanism 79. In the embodiment shown in FIG. 7, the guide slot 72 of the proximal member 61 runs non-parallel to or inclined at an angle to the longitudinal axis 38 of the instrument 30 and the guide slot 75 of the distal member 62 runs non-parallel to or inclined at an angle to the end effector axis 76. In contrast, FIG. 14 shows a swivel mechanism 79 in which one of the guide slots 72, 75 runs parallel to one of the axes 38, 76; in this case then, the guide slot 75 of the distal member 62 runs parallel to the end effector axis 76.

In contrast to FIG. 7, FIG. 15 shows a swivel mechanism 79 in which the bolts 73 and 74 are arranged in one member 62 and the guide slots 72 and 75 are arranged in the other member 61. In this variant, the two bolts 73 and 74 are thus always spaced at the same distance from one another.

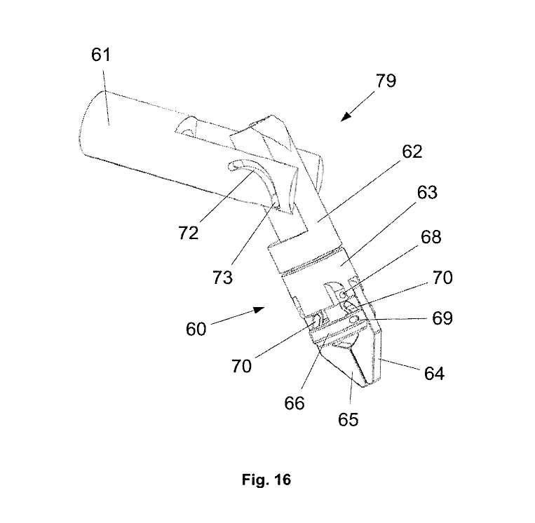

FIG. 16 shows a swivel mechanism 79 with only one guide slot 72 and only one bolt 73. Since in this case the bolt 73 is wider than in FIG. 7, it can be supported non-rotatably against the guide slot 72 on its own, i.e. the second slotted guide system for support of the torque of the distal member 62 on the proximal member 61 can thus be omitted.

### REFERENCE NUMBERS

1 fixing element
2 articulated joint
3 articulated joint
4 articulated joint
5 arm element
6 arm element
7 input device
8 drive unit
9 flange
10 robot
11 motor
12 motor
13 motor
14 motor
15 housing
16 axis
17 bearing
18 drive module
19 operating unit
20 mounting segment
21 first magnetic ring
22 second magnetic ring
23 third magnetic ring
24 fourth magnetic ring
25 magnet
26 (worm) gear
27 worm
28 gear ring
29 roller bearing
30 instrument
31 fourth wheel
32 first wheel
33 second wheel
34 third wheel
35 (not assigned)
36 ferromagnetic body
37 handle
38 longitudinal axis
39 limit stop
40 limit stop
41 second shaft
42 first shaft
43 channel
44 shaft sleeve
45 contact element
46 base element
47 (roller) bearing
48 groove
49 bearing
50 tongue
51 sleeve
52 sleeve
53 inner thread
54 groove
55 tongue
56 outer thread
57 ejector
58 holding element
59 barrier
60 end effector
61 proximal member
62 distal member
63 base
64 first gripper
65 second gripper
66 actuating member
67 screw thread
68 gripper axis
69 bolt
70 guide slot
71 bearing
72 guide slot
73 bolt
74 bolt
75 guide slot
76 end effector axis
77 drive element
78 swivel axis
79 swivelling mechanism
80 cut-out

Modifications and substitutions by one of ordinary skill in the art are considered to be within the scope of the present invention, which is not to be limited except by the following claims.

1. A drive unit (8) comprising:
at least one first drive module (18) comprising a motor (12), and a first wheel (32) driven in a rotary manner around an axis (16) by the at least one first drive module (18),
wherein the at least one first drive module (18) further comprises a first magnetic ring (22) surrounding the first wheel (32), said magnetic ring (22) coupled to the first wheel (32) in a magnetically force-transmitting manner; and
wherein said first magnetic ring (22) is mechanically coupled to the motor (12) in a force-transmitting manner.

2. The drive unit (8) according to claim 1, further including a plurality of permanent magnets (25) distributed around a circumference of at least one of the first wheel (32) and of the first magnetic ring (22), for coupling said first magnetic ring (22) to the first wheel (32) in said magnetically force-transmitting manner.

3. The drive unit (8) according to claim 1, characterised in that the mechanically force-transmitting connection between said first magnetic ring (22) and said motor (12) comprises a worm gear (26).

4. The drive unit (8) according to claim 1, further including at least a second drive module (18), arranged coaxially with the axis (16), said at least a second drive module (18) further including a second magnetic ring (23), driven in a rotary manner by a second motor (13), and by a second wheel (33) surrounded by said second magnetic ring (23).

5. The drive unit (8) according to claim 4, wherein the first wheel (32) is connected in a mechanically force-transmitting manner with a shaft (42) which extends through the second wheel (33).

6. The drive unit (8) according to claim 5, wherein a connection of the shaft (42) with the first wheel (32) is rotationally fixed in a form-locking manner and yet axially moveable.

7. The drive unit (8) according to claim 5, wherein the mechanically force-transmitting manner connection of one of the first and second wheels (32, 33) with the shaft (42) is a threaded connection.

8. The drive unit (8) according to claim 4, wherein the at least a first and at least a second drive modules (18) are arranged at intervals along the axis (16).

9. The drive unit (8) according to claim 8, wherein the at least a first and at least a second drive modules (18) include an intermediate space between the first and second magnetic rings (22, 23) and the first and second wheels (32, 33), and wherein the intermediate spaces of the at least a first and at least a second drive modules (18) align axially with one another.

10. The drive unit (8) according to claim 9, further including a germ-impermeable barrier (59) extending through the intermediate spaces of the at least a first and at least a second drive modules (18).

11. The drive unit (8) according to claim 4, characterised in that each drive module (18) includes a mounting segment (20) in which the magnetic ring (22, 23) of a respective drive module (18) is held by at least one roller bearing (29).

12. The Drive unit (8) according to claim 11, wherein the first and second magnetic rings (22, 23) include a gear ring (28), and wherein an outer diameter of said gear ring (28) is larger than that of the roller bearing (29).

13. The drive unit (8) according to claim 11, wherein the mounting segments (20) of a plurality of drive modules (18) are plug-connected with one another.

14. The drive unit (8) according to claim 4, wherein the first and second wheels (32, 33) surrounded by the first and second magnetic rings (22, 23) respectively are connected to form assembly which is accommodated removably in the first and second magnetic rings (22, 23).

15. The drive unit (8) according to claim 14, wherein the first and second wheels (32, 33) are connected with one another in a rotatable and axially immovable manner through roller bearings (47).

16. The drive unit (8) according to claim 14, wherein the assembly includes two contact elements (45, 46) disposed between which the first and second wheels (32, 33), and which contact elements (45, 46) are fixed radially to a housing (15) accommodating the first and second magnetic rings (22, 23).
